(19) United States
(12) Patent Application Publication
(10) Pub. No.: US 2011/0218191 A1
(43) Pub. Date: Sep. 8, 2011

(54) USE OF MELOXICAM FOR THE LONG TERM-TREATMENT OF KIDNEY DISORDERS IN CATS

(75) Inventor: Laura JOHNSTON, Ingelheim am Rhein (DE)

(73) Assignee: BOEHRINGER INGELHEIM VETMEDICA GMBH, Ingelheim am Rhein (DE)

(21) Appl. No.: 13/036,176

(22) Filed: Feb. 28, 2011

(30) Foreign Application Priority Data
Mar. 3, 2010 (EP) 10155400.4

(51) Int. Cl. A61K 31/5415; C07D 417/12; A61P 29/00; A61P 13/12

(52) U.S. Cl. 514/226.5; 544/49

(57) ABSTRACT

The invention is directed to a formulation containing NSAIDs or a pharmacologically acceptable salt thereof of and one or more vehicles for the treatment of kidney diseases in cats. Serum creatinine concentrations increase less over time following treatment with NSAID compared to untreated cats.

### RELATED APPLICATIONS

This application relates to and claims priority to European Patent Application No. 10155400.4, which was filed Mar. 3, 2010, the teachings and contents of which are incorporated herein by reference in their entirety. All applications are commonly owned.

### BACKGROUND OF THE INVENTION

A. Field of the Invention

The present invention is directed to the long term use of meloxicam to treat kidney diseases in cats.

B. Description of the Related Art

Chronic kidney disease (CKD) and chronic musculoskeletal diseases, such as osteoarthritis (OA) are common in elderly cats and often coexist. These conditions affect the quality of life of cats and often require treatment. Meloxicam is a COX 2 preferential NSAID of the oxicam family. It is currently the only NSAID molecule licensed for long-term use in the cat. However, impaired kidney function is listed as a contraindication or warning on NSAID data sheets. Chronic kidney diseases are very common in cats. Prevalence of renal disease in cats is considered to increase with age

Meloxicam was licensed for long-term use in cats in 2007 at an oral dose of 0.1 mg/kg on day 1 followed by 0.05 mg/kg. However, there is nothing in the art that indicates that the use of meloxicam is appropriate for long-term treatment of felines to treat chronic kidney/renal diseases. It is reported by Gunew that feline suffering from osteoarthritis can be treated with meloxicam in a concentration range between 0.01-0.03 mg/kg. (Gunew et al., Long-term safety, efficacy and palatability of oral meloxicam at 0.01-0.03 mg/kg for treatment of osteoarthritic pain in cats. Journal of Feline Medicine and Surgery 2008, 10, 235-241, hereby incorporated by reference) The trials were completed after a mean treatment duration of 5.8 months. However, this study does not include any comparable placebo product or objective efficacy measures, only subjective efficacy measurements.

Gunew further investigated the creatinine values of cats following treatment with meloxicam. A short-term (23 days) concept study performed by Clarke & Bennett has also shown that a daily meloxicam dosage of 0.05 mg/cat can be used to treat osteoarthritis. (Clarke & Bennett, Feline osteoarthritis: a prospective study of 28 cases. Journal of Small Animal Practice 2006, 47, 439-445, hereby incorporated by reference) This study does not include any thorough investigation and assessment of meloxicam treatment over a longer period of time.

Therefore, it is an object of the present invention to develop a long-term treatment of kidney/renal diseases in cats, especially aged cats

### SUMMARY OF THE INVENTION

The present invention provides compositions and related methods that overcome deficiencies in the art. The compositions and methods provide for the use of a non-steroidal anti-inflammatory drug (NSAID) or a pharmacologically acceptable salt thereof for use in the treatment of kidney diseases, including but not limited to chronic kidney diseases, in cats. Generally the cats using the compositions of the present invention will have elevated creatinine levels.

Exemplary compositions of the invention comprise meloxicam or a pharmacologically acceptable salt thereof.

Those of skill in the art will understand that the compositions used herein may incorporate known injectable, physiologically acceptable sterile solutions. For preparing a ready-to-use solution for parenteral injection or infusion, aqueous isotonic solutions, e.g. saline or plasma protein solutions, are readily available. In addition, the compositions of the present invention may include pharmaceutical- or veterinary-acceptable carriers, diluents, isotonic agents, stabilizers, thickeners, preservatives, solublizers, buffers, or pH adjusters.

The preferred treatment of such kidney diseases is a long term treatment, generally for a period of about 6 to over 40 months. The dosage range is with the expertise of the prescribing veterinarian. But may be in the range of about 0.01 and about 0.075 mg/kg, and more preferably in the range of about 0.02 and about 0.06 mg/kg daily and a most preferred daily dosage of about 0.05 mg/kg.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

It has been found that non-steroidal anti-inflammatory drugs (NSAID) such as, but not limited to, meloxicam may be used for a long-term treatment of renal diseases in cats, especially aged cats.

According to the invention, the pharmaceutically active substance, for the long-term treatment of renal diseases in cats, is a NSAID. Preferably, the NSAID is an active substance of the following categories: propionic acid derivatives, acetic acid derivatives, fenamic acid derivatives, biphenylcarboxylic acid derivatives, cyclooxygenase (COX) inhibitor of the oxicam-type/acid enolcarboxamides, diaryl heterocycles with methylsulphonyl or aminosulphonyl substituents and acid sulphonamides.

The following active substances are mentioned as examples of propionic acid derivatives, although this list should not be regarded as limiting this category of active substance: ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid and fluprofen or the pharmaceutically acceptable salts thereof.

Examples of acetic acid derivatives include the following active substances, although the list does not constitute any restriction of this category of active substance: indomethacin, sulindac, tolmetin, zomepirac, nabumetone, diclofenac, fenclofenac, alclofenac, bromfenac, ibufenac, aceclofenac, acemetacin, fentiazac, clidanac, etodolac and oxpinac or the pharmaceutically acceptable salts thereof.

The following active substances are mentioned as examples of fenamic acid derivatives, although the list does not constitute a limitation to this category of active substance: mefenamic acid, meclofenamic acid, flufenamic acid, nifluminic acid and tolfenamic acid or the pharmaceutically acceptable salts thereof.

Examples of biphenylcarboxylic acid derivatives include the following active substances, although the list does not constitute a limitation of this category of active substance: diflunisal and flufenisal or the pharmaceutically acceptable salts thereof.

The following are examples of a cyclooxygenase (COX) inhibitor of the oxicam-type/acid enolcarboxamides, such as meloxicam, piroxicam, lornoxicam, tenoxicam, droxicam, isoxicam, preferably meloxicam, or the pharmaceutically acceptable salts thereof, although the list does not constitute a restriction to this category of active substance.

Nimesulide is mentioned by way of example of an acid sulphonamide, but should not constitute a restriction to this category of active substances.

Particularly preferred according to the invention are those which contain as active substance an acid enolcarboxamide/oxicam type such as piroxicam, tenoxicam, lornoxicam and meloxicam or the pharmaceutically acceptable salts thereof, especially preferred is meloxicam.

The present invention provides non-steroidal anti-inflammatory drugs (NSAID) or a pharmacologically acceptable salt thereof for use in the treatment of chronic kidney diseases in cats. Preferably, the present invention provides a NSAID such as cyclooxygenase (COX) inhibitor of the oxicam-type/acid enolcarboxamides, preferably meloxicam or the pharmaceutically acceptable salts thereof for the treatment of chronic kidney diseases in cats. It further provides the use of a non-steroidal anti-inflammatory drugs or a pharmacologically acceptable salt thereof, such as cyclooxygenase (COX) inhibitor of the oxicam-type/acid enolcarboxamides, preferably meloxicam or the pharmaceutically acceptable salts thereof, for preparing a veterinary medical composition for the treatment of kidney diseases in cats, preferably chronic kidney diseases. The NSAID includes but is not limited to an oxicam-type compound, preferably meloxicam or a pharmacologically salt thereof. According to the invention the pharmacologically acceptable meloxicam salt preferably comprises the meglumine, potassium or ammonium salt, even more preferred the meloxicam meglumine salt.

Treatment with a composition comprising NSAID such as cyclooxygenase (COX) inhibitor of the oxicam-type/acid enolcarboxamides, preferably meloxicam or the pharmaceutically acceptable salts thereof is a long-term treatment of kidney diseases in cats. The kidney diseases are preferably chronic. The treatment improves renal function that can be monitored by measuring levels of serum creatinine. Creatinine levels in cats with CKD are elevated. These creatinine levels will increase less over time following treatment with said NSAID. The IRIS 2006 staging of CKD as shown in Table 1 defines and classifies the elevated plasma creatinine concentration. Treatment with such a formulation will not accelerate already existing renal dysfunctions/kidney diseases in older cats, nor initiate any kidney/renal disease but will in fact decrease elevated creatinine values in cats with kidney/renal diseases.

TABLE 1
IRIS 2006 Staging of CKD

| | Plasma | |
|---|---|---|
| | creatinine | |
| Stage | μmol/l) Cats | Comments |
| 1 | <140 | Non-azotemic |
| | | Some other renal abnormality present e.g. |
| | | inadequate concentrating ability without |
| | | identifiable non-renal cause; abnormal renal |
| | | palpation and/or abnormal renal imaging |
| | | findings; proteinuria of renal origin; abnormal |
| | | renal biopsy results |
| 2 | 140-249 | Mild renal azotemia [lower end of the range lies |
| | | within the reference range for many labs but |
| | | the insensitivity of creatinine as a screening |
| | | test means that animals with creatinine values |
| | | close to the upper limit of normality often |
| | | have excretory failure] |
| | | Clinical signs usually mild or absent |
| 3 | 250-439 | Moderate renal azotaemia |
| | | Many systemic clinical signs may be present |
| 4 | >440 | Severe renal azotaemia |
| | | Many extra-renal clinical signs present |

The present invention further provides a formulation containing a NSAID such as cyclooxygenase (COX) inhibitor of the oxicam-type/acid enolcarboxamides, preferably meloxicam or pharmaceutically acceptable salts thereof, for the treatment of chronic kidney diseases in cats that essentially consists of a non-steroidal anti-inflammatory drug, water, optionally one or more additives selected from the group consisting of buffers, solubilizers, preservatives and optionally thickeners. Said formulation comprises a NSAID such as meloxicam or a pharmacologically acceptable salt thereof. Furthermore the present invention provides the use of a formulation containing NSAID, such as cyclooxygenase (COX) inhibitor of the oxicam-type/acid enolcarboxamides, preferably meloxicam or pharmaceutically acceptable salts thereof, for preparing a veterinary composition for the treatment of kidney diseases in cats that essentially consists of a non-steroidal anti-inflammatory drug, water, optionally one or more additives selected from the group consisting of buffers, pH adjusters, solubilizers, preservatives and optionally thickeners.

In another aspect, the invention relates to administration of a formulation comprising meloxicam and other excipients as defined herein for use in the treatment of kidney diseases and to decelerate said kidney diseases, preferably chronic kidney diseases.

According to the invention, the formulation preferably contains a NSAID such as an oxicam-type compound, preferably meloxicam, as a base or a pharmaceutically acceptable salt thereof. Preferably, the salt of meloxicam is selected from the group consisting of meglumine, sodium, potassium or ammonium salt, most preferably the meloxicam meglumine salt.

Other ingredients of the solution or suspension comprise commonly known agents for suspensions or solutions such as suspending agents, preservatives, flavoring agents, ph adjusters and solvents such as for example water that are used for said formulations.

Suspending agents used may be for example organic hydrocolloid forming agents such as cellulose ether and/or silicon dioxide, preferably hydroxyethyl cellulose, hydroxypropyl cellulose, hydroxypropylmethyl cellulose and/or silicon dioxide or colloidal anhydrous silica, preferably colloidal anhydrous silica and/or hydroxyethyl cellulose.

Preservatives used may be for example benzoic acid or any derivatives or salts thereof, preferably sodium benzoate.

Flavoring agents used may be for example sugar alcohols such as glycerol, sorbitol, mannitol, xylitol or artificial sweeteners such as saccharin or any of its salt, cyclamate, aspartame, sucralose, taumatin, or any of their salts, acesulfam-potassium, aqueous solutions thereof, or mixtures thereof, preferably sorbitol, glycerol saccharin or sodium saccharin and glycerol. Other flavoring agents may be artificial aromas such as an artificial fruit or meat aroma as for example honey, strawberry, raspberry, or beef or fish flavor, preferably honey.

The pH adjusters used may be for example sodium dihydrogen phosphate dihydrate/citric acid monohydrate buffer, glycine/HCl, K-hydrogen phthalate/HCl, citric acid/phosphate, citrate-phosphate-borate/HCl or Britton-Robinson buffer, mixtures thereof or mixtures with other physiologically acceptable liquids such as glycerol or optionally aqueous solutions of sugar alcohols, preferably sodium dihydrogen phosphate dihydrate and citric acid monohydrate.

Preferably the formulation used for the treatment of kidney diseases in cats comprises meloxicam as the active ingredient, highly dispersed silicon dioxide, hydroxyethyl cellulose, sorbitol solution (non-crystalline), glycerol, xylitol, sodium dihydrogen phosphate dihydrate, citric acid monohydrate, saccharin sodium crystals, sodium benzoate and flavor and with purified water.

In another aspect, the invention preferably relates to a formulation used for the treatment of kidney diseases in cats comprising meloxicam, sodium benzoate, colloidal anhydrous silica, hydroxyethyl cellulose, mannitol, glycerol, saccharin sodium dihydrate, xylitol, glycine, HCl, flavor and purified water.

The treatment occurs over a long-term period of at least 6 months, preferred ranges are selected from the group selected of 6 to 40 months, 10 to 40 months, 10 to 37 months, 10 to 30 months, 10 to 25 months, 10 to 20 months, 10 to 17 months, 11 to 40 months, 11 to 37 months, 11 to 30 months, 11 to 25 months, 11 to 20 months, 11 to 17 months, 12 to 40 months, 12 to 37 months, 12 to 30 months, 12 to 25 months, 12 to 20 months, 12 to 17 months, 13 to 40 months, 13 to 37 months, 13 to 30 months, 13 to 25 months, 13 to 20 months, 13 to 17 months, 14 to 40 months, 14 to 37 months, 14 to 30 months, 14 to 25 months, 14 to 20 months and 14 to 17 months.

Older cats or aged cats are herein defined as being 5 years old or older, preferably from 5 to 20 years, even more preferably from 7 to 17 years, especially preferred from 10 to 13.4 to 15.5 to 16 years. Studies have shown that 53% of cats over 7 years of age have renal diseases. Kidney/renal diseases may include acquired renal diseases such as chronic tubulo-interstitial nephritis, glomerulonephritis, pyelonephritis, amyloidosis, hydronephrosis, renal lymphoma or congenital diseases that cause kidney failure in cats such as polycystic kidney disease, renal aplasia, renal hypoplasia, renal dysplasia, amyloidosis. These may or may not be in a chronic disease state.

In another embodiment the treatment of cats may be performed in a formulation useful for cats that are 5 years or older, preferably from 5 to 20 years, even more preferably from 7 to 17 years, especially preferred from 10 to 13.4 to 15.5 to 16 years. The daily dose of the formulation is between 0.01 and 0.075 mg/kg daily, preferably from 0.01 to 0.05 mg/kg, even more preferred is from 0.01 to 0.03 mg/kg. The lowest effective dose for a median maintenance dose was found to be 0.02 mg/kg. This range can be used to treat renal diseases. Preferably, the formulation contains or essentially consists of meloxicam salt, water, optionally one or more additives selected from the group consisting of buffers, solubilizers, preservatives and optionally thickeners.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

The medical records of a feline-only practice were searched for cats with OA being treated with meloxicam during a 4 year period. A diagnosis of OA was based upon any two of the following: owner noted mobility changes and/or physical examination findings or radiographic changes. Cats included were older than 7 years and treated with meloxicam for a duration of more than 6 months. Biochemistry, urinalysis and body weight were regularly monitored. The progression of renal disease in the aged non-renal and renal group treated was compared to age matched and IRIS matched untreated controls from the same clinic. IRIS staging of CKD follows specific guidelines on the diagnosis and assessment of progression of renal disease in small animals as described below. Animals are being divided into three categories according to the stage of their disease, see Table 1 (iris-kidney.com).

Surprisingly the results show that a maintenance dose of 0.02 mg/kg meloxicam does not hasten progression of renal disease in aged cats belonging to IRIS stage 1-3 renal disease but actually delays progression of the creatinine values and thus showing signs of a delayed deterioration of renal function. Therefore meloxicam can be used as a treatment for cats with renal diseases.

### Materials and Methods

The database of a feline-only practice in suburban Melbourne was searched for cats which had been treated for chronic musculoskeletal diseases with meloxicam (Metacam® oral suspension, Boehringer Ingelheim) during a 4 year period. The diagnosis of osteoarthritis or spondylosis deformans had been made based upon any two of the following: owner noted mobility changes, physical examination findings or radiographic changes. The inclusion criteria included cats greater than 7 years old which had been treated continuously with meloxicam for a duration of more than 6 months and which had complete medical records available for review. In addition, cats were only included if serum biochemistry, urine analysis and body weight had been regularly monitored.

Young cats were excluded, as were cats with no pre-treatment renal parameter measurements. In addition cats were also excluded if the owner could not be contacted to check that the cats were still receiving daily treatment with meloxicam.

Age, breed, sex, concomitant diseases and medications as well as date treatment commenced, treatment duration and daily dose of meloxicam were recorded.

The majority of serum biochemistry determination was via external reference laboratory (Normal Range: Creatinine 0.08-0.20 mmol/L) and the remainder with an in-house IDEXX biochemistry and electrolyte machine (Normal Range: Creatinine 71-212 umol/L). In house urine specific gravity was determined with a Reichert Vet360 refractometer. Urine specific gravity was regularly compared to that measured by the same reference laboratory. Urine sediment examination and dipstick determination was carried out in-house with necessary urine cultures and urine protein. Urine creatinine determination was carried out at the same reference laboratory.

The presence or absence of pre-treatment renal disease and staging of the renal disease was carried out using plasma creatinine and urine specific gravity.

The treated cats were then subdivided into two groups: the renal treated group that belonged to IRIS stage 1-3, and the non-renal treated group that has no identifiable renal disease pre-treatment. Urine specific gravity, serum creatinine and bodyweight were used as indicators of renal disease progression. The progression of renal disease was then compared to age and IRIS matched untreated controls were then randomly identified from the database of the same clinic.

The cats were then subdivided into four groups; two meloxicam treated groups and two comparator groups, to form a two-way case-controlled retrospective study.

Group A: Aged cats with chronic kidney disease (CKD), treated with meloxicam (i.e. those with IRIS stage 1-3 pre-treatment)

Group B: Aged cats without chronic kidney disease (no-CKD) treated with meloxicam (i.e. those with no identifiable kidney disease pre-treatment).

Group C: Aged cats with CKD, not receiving meloxicam (i.e. those with IRIS stage 1-3)

Group D: Aged cats without CKD, not receiving meloxicam (i.e. those with no identifiable kidney disease pre-treatment)

Statistical Analysis

The median age of the renal and non-renal treated cats, median treatment duration and median maintenance dose was calculated.

The progression of renal disease in the non-renal treated group was compared to the age matched untreated controls. The progression of renal disease in the treated renal-diseased group was compared to age matched and IRIS matched untreated controls from the same clinic. Statistical analysis was carried out using a time adjusted area-under-the-curve (AUC) changes from baseline time 0 until the last recorded value (n).

$${AUC}_{( 0 - n ) , adj} = \frac{{AUC}_{( 0 - n )}}{{t}_{n} - {t}_{0}} = \frac{{\sum }_{i = 0}^{n} \frac{{C}_{i} + {C}_{i + 1}}{2} ( {t}_{i + 1} - {t}_{i} )}{{t}_{n} - {t}_{0}}$$

t₀: time point of first measurement (baseline)

tₙ: time point of last measurement

Cᵢ: difference of parameter concentration at time point i=0, . . . , n to baseline

The Wilcoxon rank-sum-test was used to compare the groups.

With this nonparametric test the distribution of the adjusted AUC of two groups was compared regarding the location. Under the null hypothesis it is assumed that there is no location shift in the distributions of the two treatment groups. If the resulting p-value is lower than the two-sided significance level of 5% the null hypothesis is rejected.

### Results

Out of a total database of 3016 cats, 214 cats which had been treated with Metacam oral suspension were identified. Of these, 38 cats met the inclusion criteria for the meloxicam-treated group (A+B). 22 cats of these cats (58%) had IRIS stage 1-3 CKD prior to treatment, whereas 8 cats were categorised as IRIS stage 1, 13 cats belonged to stage 2 and 1 cat was classified as belonging to stage 3. A further 16 cats had no identifiable renal disease prior to treatment.

The median age of the renal treated group (A) was 15.5 years and the non-renal treated group (B) was 13.4 years.

The median treatment duration was 467 days in the renal group (A) and 327 days in the non-renal group (B). After dose titration to the lowest effective dose, the median maintenance dose was 0.02 mg/kg daily in both the renal treated and non-renal treated groups. There were no differences in the progression of renal parameters in the renal group treated with meloxicam versus the age and IRIS matched untreated renal group or the non-renal group treated with meloxicam versus the non-renal group not treated with meloxicam.

Two renal-treated cats were excluded from analysis of creatinine changes from baseline as their pre-treatment sample was not carried out within 3 days of the start of treatment. There were no statistically significant differences from baseline in body weight in the CKD-meloxicam group (A) versus the age and IRIS matched, untreated CKD group (C) or between the no-CKD meloxicam group (B) versus the untreated group no-CKD (D). Mean serum creatinine concentration increased less over time in the meloxicam treated CKD group (A) compared to the untreated CKD group (C). Furthermore, there was no significant difference in the progression of creatinine in the meloxicam treated no-CKD treated group (B) and the untreated no-CKD.

### Example 1

Preferably a meloxicam formulation is used such as for example but not limiting to Metacam oral suspension for cats. According to the invention the following formulations examples may be used but not limited to:

### Example 1

0.05 g meloxicam, 1 g highly dispersed silicon dioxide, 0.1 g hydroxyethyl cellulose, 35 g 70% sorbitol solution (non-crystalline), 12.8 g 85% glycerol, 15 g mannitol, 1.5 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 2

0.15 g meloxicam, 1 g highly dispersed silicon dioxide, 0.1 g hydroxyethyl cellulose, 35 g 70% sorbitol solution (non-crystalline), 12.8 g 85% glycerol, 15 g mannitol, 1.5 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 3

0.05 g meloxicam, 1 g colloidal anhydrous silica, 0.1 g hydroxyethyl cellulose, 35 g 70% sorbitol solution (non-crystalline), 12.8 g 85% glycerol, 15 g mannitol, 1.5 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 4

0.15 g meloxicam, 1 g colloidal anhydrous silica, 0.1 g hydroxypropyl cellulose, 35 g 70% sorbitol solution (non-crystalline), 12.8 g 85% glycerol, 15 g mannitol, 1.5 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 5

0.05 g meloxicam, 1 g highly dispersed silicon dioxide, 0.1 g hydroxyethyl cellulose, 35 g 70% sorbitol solution (non-crystalline), 12.8 g 85% glycerol, 15 g xylitol, 2 g sodium dihydrogen phosphate dihydrate, 0.12 g citric acid monohydrate, 0.010 g saccharin sodium crystals, 0.15 g sodium benzoate and 0.15 g flavour such as honey, strawberry, raspberry, beef or fish. The mixture is made up to a final volume of 100 ml with purified water.

### Example 6

0.15 g meloxicam, 1 g highly dispersed silicon dioxide, 0.1 g hydroxyethyl cellulose, 35 g 12.70 70% sorbitol solution (non-crystalline), 12.8 g 85% glycerol, 15 g xylitol, 2 g sodium dihydrogen phosphate dihydrate, 0.12 g citric acid monohydrate, 0.01 g saccharin sodium crystals, 0.15 g sodium benzoate and flavour such as honey, strawberry, raspberry, beef or fish. The mixture is made up to a final volume of 100 ml with purified water.

### Example 7

0.05 g meloxicam, 1.5 g highly dispersed silicon dioxide, 0.1 g hydroxyethyl cellulose, 40 g 70% sorbitol solution (non-crystalline), 10 g 85% glycerol, 5 g xylitol, 0.2 g sodium dihydrogen phosphate dihydrate, 0.1 g citric acid monohydrate, 0.030 g saccharin sodium crystals, 0.20 g sodium benzoate and 0.05 g flavour such as honey, strawberry, raspberry, beef or fish. The mixture is made up to a final volume of 100 ml with purified water.

### Example 8

0.15 g meloxicam, 1.5 g highly dispersed silicon dioxide, 0.1 g hydroxyethyl cellulose, 40 g 70% sorbitol solution (non-crystalline), 10 g 85% glycerol, 5 g xylitol, 0.2 g sodium dihydrogen phosphate dihydrate, 0.1 g citric acid monohydrate, 0.030 g saccharin sodium crystals, 0.20 g sodium benzoate and 0.05 g flavour such as honey, strawberry, raspberry, beef or fish. The mixture is made up to a final volume of 100 ml with purified water.

### Example 9

0.05 g meloxicam, 0.5 g highly dispersed silicon dioxide, 0.5 g hydroxyethyl cellulose, 20 g 70% sorbitol solution (non-crystalline), 20 g 85% glycerol, 10 g xylitol, 3 g sodium dihydrogen phosphate dihydrate, 0.1 g citric acid monohydrate, 0.020 g saccharin sodium crystals, 0.10 g sodium benzoate and 0.05 g flavour such as honey, strawberry, raspberry, beef or fish. The mixture is made up to a final volume of 100 ml with purified water.

### Example 10

0.15 g meloxicam, 0.5 g highly dispersed silicon dioxide, 0.5 g hydroxyethyl cellulose, 20 g 70% sorbitol solution (non-crystalline), 20 g 85% glycerol, 10 g mannitol, 3 g glycine, 0.1 g HCl, 0.020 g aspartame, 0.10 g sodium benzoate and 0.05 g flavour such as honey, strawberry, raspberry, beef or fish. The mixture is made up to a final volume of 100 ml with purified water.

### Example 11

0.05 g meloxicam, 1 g colloidal anhydrous silica, 0.1 g hydroxyethyl cellulose, 35 g 70% sorbitol solution (non-crystalline), 12.8 g 85% glycerol, 15 g mannitol, 0.2 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 12

0.15 g meloxicam, 1 g colloidal anhydrous silica, 0.1 g hydroxyethyl cellulose, 35 g 70% sorbitol solution (non-crystalline), 12.8 g 85% glycerol, 15 g mannitol, 0.2 g glycine, 0.12 g HCl, 0.010 g aspartame, 0.15 g sodium benzoate and 0.15 g flavour such as honey, strawberry, raspberry, beef or fish. The mixture is made up to a final volume of 100 ml with purified water.

### Example 13

0.05 g meloxicam, 1.5 g highly dispersed silicon dioxide, 0.05 g hydroxyethyl cellulose, 45 g 70% sorbitol solution (non-crystalline), 10 g 85% glycerol, 10 g xylitol, 3 g sodium dihydrogen phosphate dihydrate, 0.15 g citric acid monohydrate, 0.010 g saccharin sodium crystals, 0.15 g sodium benzoate and 0.15 g flavour such as honey, strawberry, raspberry, beef or fish. The mixture is made up to a final volume of 100 ml with purified water.

### Example 14

0.15 g meloxicam, 1.5 g highly dispersed silicon dioxide, 0.05 g hydroxyethyl cellulose, 45 g 70% sorbitol solution (non-crystalline), 10 g 85% glycerol, 10 g xylitol, 3 g sodium dihydrogen phosphate dihydrate, 0.15 g citric acid monohydrate, 0.010 g saccharin sodium crystals, 0.15 g sodium benzoate and flavour such as honey, strawberry, raspberry, beef or fish. The mixture is made up to a final volume of 100 ml with purified water.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

What is claimed is:

1. Non-steroidal anti-inflammatory drug (NSAID) or a pharmacologically acceptable salt thereof for use in the treatment of chronic kidney diseases in cats.

2. A non-steroidal anti-inflammatory drug or a pharmacologically acceptable salt thereof for use in the treatment of kidney diseases in cats.

3. A non-steroidal anti-inflammatory drug or a pharmacologically acceptable salt thereof according to claim 1, wherein the NSAID is meloxicam or a pharmacologically acceptable salt thereof.

4. A non-steroidal anti-inflammatory drug or a pharmacologically acceptable salt thereof according to claim 2, wherein the NSAID is meloxicam or a pharmacologically acceptable salt thereof.

5. A non-steroidal anti-inflammatory drug or a pharmacologically acceptable salt thereof according to claim 1, wherein the treatment is a long term treatment.

6. A non-steroidal anti-inflammatory drug or a pharmacologically acceptable salt thereof according to claim 2, wherein the treatment is a long term treatment.

7. A non-steroidal anti-inflammatory drug or a pharmacologically acceptable salt thereof according to claim 5, wherein said treatment is a long term treatment over 6 to 40 months.

8. A non-steroidal anti-inflammatory drug or a pharmacologically acceptable salt thereof according to claim 6, wherein said treatment is a long term treatment over 6 to 40 months.

9. A non-steroidal anti-inflammatory drug or a pharmacologically acceptable salt thereof according to one of claim 1, wherein the daily dose is from 0.01 and 0.075 mg/kg.

10. A non-steroidal anti-inflammatory drug or a pharmacologically acceptable salt thereof according to one of claim 2, wherein the daily dose is from 0.01 and 0.075 mg/kg.

11. A non-steroidal anti-inflammatory drug or a pharmacologically acceptable salt thereof according to one of claim 1, wherein the daily dose is from 0.02 to 0.06 mg/kg.

12. A non-steroidal anti-inflammatory drug or a pharmacologically acceptable salt thereof according to one of claim 2, wherein the daily dose is from 0.02 to 0.06 mg/kg.

13. A non-steroidal anti-inflammatory drug or a pharmacologically acceptable salt thereof according to one of claim 1, wherein the daily dose is 0.05 mg/kg.

14. A non-steroidal anti-inflammatory drug or a pharmacologically acceptable salt thereof according to one of claim 2, wherein the daily dose is 0.05 mg/kg.

15. A non-steroidal anti-inflammatory drug or a pharmacologically acceptable salt thereof according to claim 1, wherein in that the cats have elevated creatinine levels.

16. A non-steroidal anti-inflammatory drug or a pharmacologically acceptable salt thereof according to claim 2, wherein in that the cats have elevated creatinine levels.

17. A formulation containing a NSAID or a pharmacologically acceptable salt thereof for the treatment of chronic kidney diseases in cats, wherein said formulation essentially consists of a non-steroidal anti-inflammatory drug, water, optionally one or more additives selected from the group consisting of buffers, pH adjusters, solubilizers, preservatives and optionally thickeners.

18. A formulation according to claim 17, wherein the NSAID is meloxicam or a pharmacologically acceptable salt thereof.

19. A method of treating kidney disease in cats comprising administering an effective amount of a non-steroidal anti-inflammatory drug (NSAID) or a pharmacologically acceptable salt to a cat in need of said treatment.

20. A method according to claim 19, wherein said cat has elevated creatinine levels.

21. A method according to claim 19, wherein the treatment is long term.

22. A method according to claim 21, wherein said treatment is over 6 to 40 months.

23. A method according to claim 19, wherein the daily dose is from 0.02 to 0.06 mg/kg.

24. A method according to claim 19, wherein the daily dose is from 0.01 and 0.075 mg/kg.

25. A method according to claim 19, wherein the daily dose is about 05 mg/kg.
